(19) United States
(12) Patent Application Publication
(10) Pub. No.: US 2015/0353885 A1
(43) Pub. Date: Dec. 10, 2015

(54) METHOD TO COUNTER-SELECT CELLS OR ORGANISMS BY LINKING LOCI TO NUCLEASE COMPONENTS

(71) Applicant: CELLECTIS, Paris (FR)

(72) Inventor: David Sourdive, Levallois (FR)

(21) Appl. No.: 14/769,350

(22) PCT Filed: Feb. 21, 2014
(86) PCT No.: PCT/IB2014/059156

§ 371 Date: Aug. 20, 2015

Related U.S. Application Data
(60) Provisional application No. 61/767,593, filed on Feb. 21, 2013.

(51) Int. Cl. C12N 5/071

(52) U.S. Cl. CPC C12N 5/0608

(57) ABSTRACT

The present invention relates to the field of genetic selection, where particular genetic traits or loci combinations are sought in a progeny resulting from genetic breeding. The invention provides genetic engineering solutions to select or counter-select the occurrence of genetic events.

### FIELD OF THE INVENTION

The present invention relates to the field of genetic selection, where particular genetic traits or loci combinations are sought in a progeny resulting from genetic breeding. The invention provides with genetic engineering solutions to select or counter-select the occurrence of genetic events.

### BACKGROUND OF THE INVENTION

One problem met in biology resides in preventing the occurrence of events (cells, organelles or organisms) characterized by a particular genetic or epigenetic combination. This issue is raised when such events grow, propagate or persist (e.g. organelle composition, gene extinction) or when their genetic composition changes (e.g. mating, hybridization, segregation). This imposes a strong limitation in obtaining genetic lineages comprising a selection of genes or traits.

Approaches to this problem currently reside mainly in:
(i) sorting the events that do not bear the unwanted genetic or epigenetic characteristics, which is often cumbersome due to the required intervention to prevent the unwanted events to persist or propagate, and in many instances impossible if the sought genetic combination does not confer any competitive advantage or selectable phenotype;
(ii) exercising a selection pressure to favor the wanted events, which is not always applicable and may lead to cells or organisms escaping the selection pressure by genetic recombination or mutations, thereby requiring to combine multiple selection pressures.

Plant genetics, in particular, remains very ponderous and time consuming due to the fact that plant genomes are very large, often comprise multiple alleles or gene copies, and breeding processes are uncertain due to pollination. Also, phenotype improvements in plants go through the maintenance of QTL (Quantitative Traits Loci), which are stretches of DNA containing or linked to genes that underlie a quantitative trait.

The present invention aims at driving the genetic selection of one or multiple combinations of genes over generations, by preventing genetic recombination to occur in selected parts of the genome.

More particularly, the invention makes use of:
(i) a nuclease component (the Toxic Nuclease(s)) that can be lethal or detrimental to the propagation, replication or reproduction of a cell, an organelle, a tissue or a whole organism (the Toxic Effect) and
(ii) a specific anti-nuclease component, which acts as an inhibitor of the nuclease activity of said specific nuclease component.

The principle lies in placing or expressing, either transiently or permanently, one or more Toxic Nuclease(s), in a cell, organelle, tissue or whole organism, in which one or more Inhibitor(s) can be active. When said Inhibitor(s) are not active, the Toxic Nuclease(s) can have its (their) Toxic Effect and leads to the elimination of progeny cells not carrying/expressing the inhibitor gene.

This invention can be used to counter-select cells, organelles, tissues or whole organisms in which said Inhibitor(s) are not active.

This provisional application contains three figures executed in color.

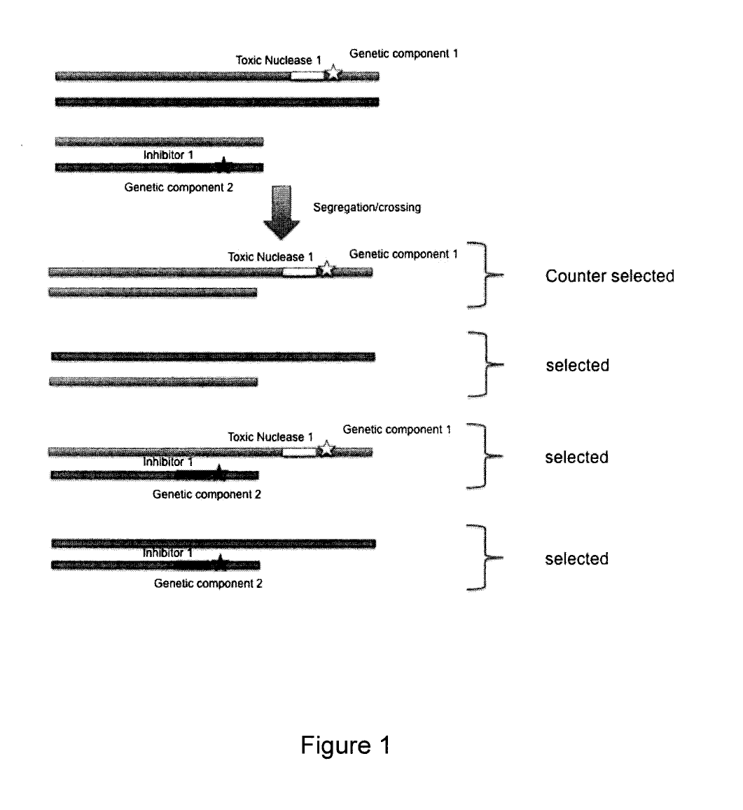

FIG. 1: selection according to the invention of progeny cells bearing the combination of the genetic loci components 1 and 2 upon segregation of parental genome (ex: meiosis).

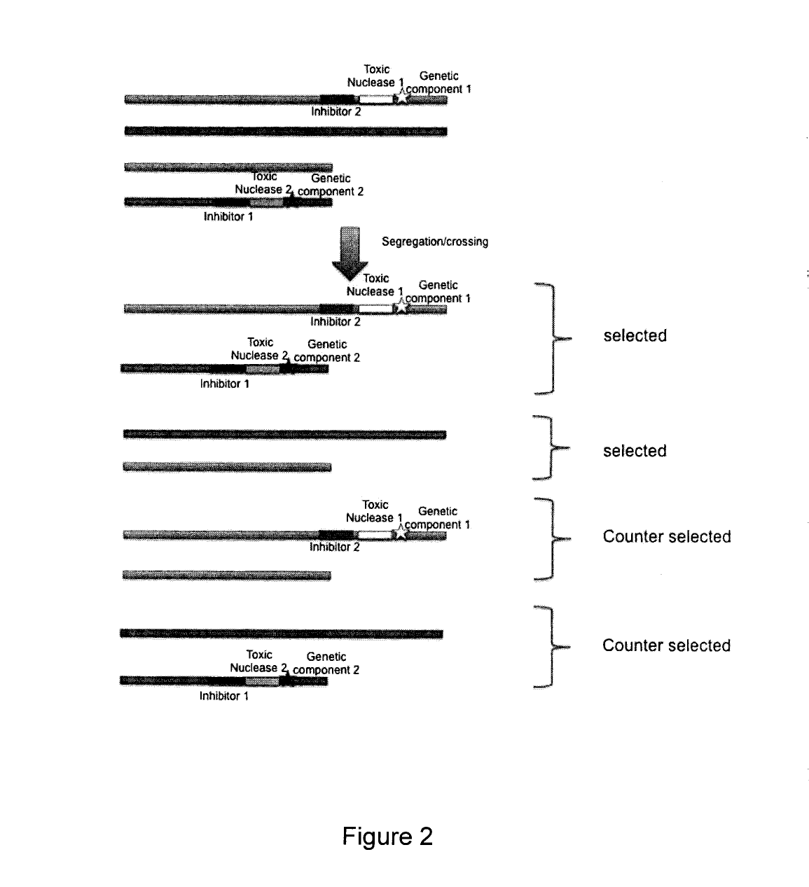

FIG. 2: selection according to the invention of progeny cells bearing the combination of the genetic loci components 1 and 2 upon segregation of parental genome (ex: meiosis) by using crossed nuclease/inhibitors couples.

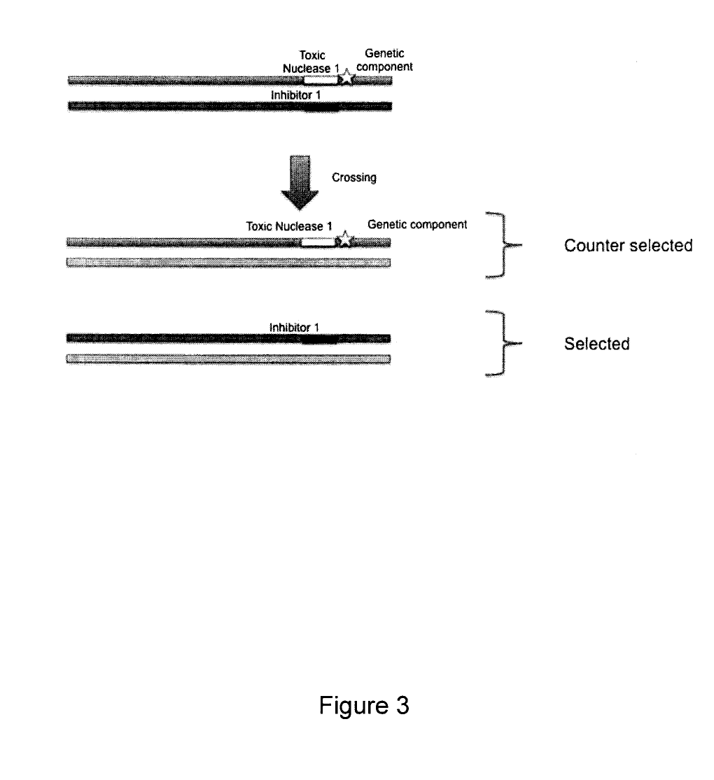

FIG. 3: Counter-selection according to the invention of progeny cells bearing only the locus comprising the undesired genetic component linked to the toxic nuclease component.

### DETAILED DESCRIPTION OF THE INVENTION

Previous patent applications have disclosed various methods for making and using specific nucleases, in particular rare-cutting endonucleases for cleaving specific nucleic acid sequences in genomes. By rare-cutting endonuclease is meant an endonuclease that has a polynucleotide recognition site of at least 12 base pairs (bp) in length, preferably from 14 to 55 bps. Such endonucleases can either be derived from natural proteins having endonuclease activity, such as homing endonucleases (WO 2004/067736), or by fusion of various nucleic acid binding polypeptides to nuclease components, such as Fok-1 or Tev-1 catalytic domains (WO2012138927). Appropriate nucleic acid binding domains that can be engineered in this respect are, for instance, Zing Finger domains (Kim et al., 1994, Chimeric restriction endonuclease, PNAS, 91:883-887), TAL effectors originating from microbes related to Xanthomonas (WO 2011/072246) or more recently MBBBD (Modular base-per-base binding domains) originating from the endosymbiotic Burkholderia rhizoxinica. More recently, a new system involving nuclease Cas9 homologues and RNaseIII (CRISPR/Cas9) has been developed from the immune system of bacterial microorganisms. In this system, the specificity of the endonuclease protein complex is addressed by specific single stranded RNAs called “guide-RNA”. This guide-RNA has the ability to hybridize the nucleic acid target sequence to be cleaved by the nuclease component Cas9 (Le Cong et al., 2013, Multiplex genome engineering using CRIPR/Cas systems, Science, 339 (6121): 819-823)

The above endonucleases can be used either as tools for gene editing, in particular to help homologous recombination to occur in the genome at a desired locus, or be used for toxic expression into cells as part of the present invention. For this later purpose, genes encoding the nucleases can be stably integrated into the genome of cells by using the first endonucleases or other recombination techniques, and the expression of the nuclease is activated to confer a genotoxic effect into said cells.

By “genotoxic effect”, it is meant a toxic effect resulting from the activity of the nuclease component expressed into the cell, the genome being the nucleic acid substrate for said nuclease. The activity of the nuclease component can be specific or not specific. When it is specific, for instance, when the nuclease is a rare-cutting endonuclease, the nuclease is active only in some parts of the genome where the nucleic acid sequences targeted by said endonuclease can be found and to the extent permitted by the epigenetic status of the nucleic acid substrate and the affinity of the endonuclease for such target sequences.

A nuclease inhibitor according to the invention designates a product of a gene that can selectively neutralize the nuclease activity of a nuclease protein or protein complex. The neutralization may originate from different mechanisms, such as for instance direct inhibition of the catalytic domain by formation of an inactive complex (ex: NucA/NuiA as reported by Ghosh et al., The Nuclease A—inhibitor complex is characterized by a novel metal ion bridge, 2007, JBC, 282(8):5682-5690), interference at the expression level (ex: expression of interference RNA against the mRNA encoding the nuclease) or neutralization of the protein by a specific antibody. With respect to CRIPR/Cas9 nuclease complex, the present invention suggests to use a molecule referred to as an anti-guideRNA, for instance a polynucleotide that can hybridize the guide-RNA to form double stranded RNA that will not be able to address specificity to Cas9, and therefore will neutralize the nuclease activity of the Cas9 complex (at least with respect to its initial target sequence).

The present invention encompasses various embodiments based on the use of the above nucleases for the purpose of improving genetic selection. The main embodiments are disclosed in the following sections without limitation:

### 1. GENETIC LINKAGE

### 1.1. Simple Hemi-Linkage

The invention can be applied to bias the genetic linkage between two genetic loci or components, by
(i) linking one or more active Inhibitor(s) to one genetic locus, or a genetic component (the First Locus), e.g. by placing the gene(s) coding for said Inhibitor(s) into said locus or next to said genetic component, and
(ii) linking one or more Toxic Nuclease(s) to a second genetic locus, or a genetic component (the Second Locus), e.g. by placing the gene(s) coding for said Toxic Nuclease(s) into said locus or next to said genetic component.

Should the Second Locus be separate from the First, the Inhibitor(s) would no longer block the Toxic Nuclease(s) from preventing the propagation of the cell or organism.

An example of simple hemi-linkage can be achieved by using a custom designed or naturally occurring DNA recognizing component that will either bind the genome in many sites (e.g. nuclease recognizing ribosome rDNA, or a repeated element in the genome) numerous enough so that their concomitant cleavage can result in cell or organelle death, or that binds the genome in critical sites (e.g. the sequence coding for the catalytic site of an essential gene), linked to a gene encoding a protein with DNA cleaving activity that can be inhibited by another peptide. Examples of such nucleases are obtained using a DNA binding component (e.g. polypeptide) fused to the NucA catalytic domain that can be inhibited by nuiA, or, to the CoIE7 catalytic domain that can be inhibited by Im7. Such pairs of nuclease activity bearing domain/specific inhibitor can also be build using existing nucleases as referred to before and antagonistic ligands that block their activity, such as antibodies or fragments thereof (including, but not limited to camelidae antibodies) or antagonistic peptides or blocking RNA that can each be encoded by a gene.

Using a nuclease targeting a repeated element in the genome can be advantageous to avoid the occurrence of a resistance: evading the Toxic Effect is very unlikely (it would take the cell/organelle/organism to mutate hundreds of Nuclease target sequence at one time, which is highly improbable). For example, a nuclease targeting rDNA in human cells would be lethal if not inhibited (e.g. I-Ppo 1)

### 1.2. Cross Linkage

The invention can also be applied to further bias the linkage between two genetic loci or components, by using two sets of Toxic Nuclease(s) and corresponding Inhibitor(s):
(i) linking one or more active Inhibitor(s) of the first set and one ore more Toxic Nuclease(s) of the second set (i.e. not inhibited by said co-linked Inhibitors of the first set), to one genetic locus or a genetic component (the First Locus)—e.g. by placing the gene(s) coding for said Inhibitor(s) and genes coding for said First Toxic Nuclease(s) into said locus or next to said genetic component—and
(ii) linking one or more active Inhibitor(s) of the second set and one ore more Toxic Nuclease(s) of the first set (i.e. not inhibited by said co-linked Inhibitors of the second set), to another genetic locus or a genetic component (the Second Locus)—e.g. by placing the gene(s) coding for said Inhibitor(s) and genes coding for said First Toxic Nuclease(s) into said locus or next to said genetic component.

Should the First and Second Loci be separate, the Inhibitor(s) would no longer block the corresponding Toxic Nuclease(s) from preventing the propagation of the cell, organelle or organism.

An example of cross linkage can be achieved by using:
(i) a custom designed or naturally occurring DNA recognizing polypeptide having a nuclease component that will either bind the genome in many sites numerous enough so that their concomitant cleavage can result in cell or organelle death, or that binds the genome in critical sites (e.g. the sequence coding for the catalytic site of an essential gene), linked to a DNA cleaving activity that can be inhibited by another peptide, such as the NucA catalytic domain that can be inhibited by nuiA, and/or
(ii) a custom designed or naturally occurring DNA recognizing polypeptide that will either bind the genome in many sites (e.g. nuclease recognizing rDNA, or a repeated element in the genome) numerous enough so that their concomitant cleavage can result in cell or organelle death, or that binds the genome in critical sites (e.g. the sequence coding for the catalytic site of an essential gene), linked to a DNA cleaving activity that can be inhibited by another peptide, such as the CoIE7 catalytic domain that can be inhibited by Im7, and optionally
(iii) placing the gene coding for the NucA-linked nuclease and the gene coding for Im7 into the First locus, and placing the gene coding for the CoIE7-linked nuclease and the gene coding for nuiA into the Second locus.

After random segregation, the two genetic components are strongly linked.

An additional feature can be combined by exercising a positive selection pressure for either or both of the linked genetic components (e.g. placing one or more positive selection markers close to either or both said genetic components). Then, the events bearing the two genetic components together will be strongly privileged in the segregation.

### 1.3. Linkage of Multiple Loci to One Locus

The invention can also be applied to bias the linkage between more than two genetic loci or components, using sets of Toxic Nuclease(s) and corresponding Inhibitor(s) by:
(i) linking one or more active Inhibitor(s) of each set to individual genetic loci, or genetic components (the Nth Locus), e.g. by placing the gene(s) coding for said Inhibitor(s) into said locus or next to said genetic component, and/or
(ii) linking one or more Toxic Nuclease(s) of every set to one last genetic locus, or a genetic component (the Last Locus), e.g. by placing the gene(s) coding for said Toxic Nuclease(s) into said locus or next to said genetic component. Alternatively, Toxic Nuclease(s) with multiple cleavage activities each inhibited by individual sets of Inhibitors can be used.

After random segregation, the Last Locus will be strongly linked to all the Nth Loci.

An additional feature can be combined by exercising a positive selection pressure for the genetic component in the Nth Locus (e.g. placing one or more positive selection markers close to said genetic component). Then, the events bearing all the N and the Last genetic components together will be strongly privileged in the segregation.

### 1.4. “Circular” Linkage of Multiple Loci

The invention can also be applied to bias the linkage between N (more than two) genetic loci or components, using N sets of Toxic Nuclease(s) and corresponding Inhibitor(s): With the sets of Toxic Nuclease(s) and corresponding Inhibitors are numbered from 1 to N, and the loci to be linked are also numbered from 1 to N:
(i) for p ranging from 1 to N-1, linking one or more active Inhibitor(s) of the set number p and a one or more Toxic Nuclease(s) of the set number p+1, to the genetic locus or genetic component number p—e.g. by placing the gene(s) coding for said Inhibitor(s) and genes coding for said Toxic Nuclease(s) into said locus or next to said genetic component—and
(ii) linking one or more active Inhibitor(s) of the set number N and a one or more Toxic Nuclease(s) of the set number 1, to the genetic locus or genetic component number N

After random segregation, the all the N loci will be strongly linked together.

An additional feature can be combined by exercising a positive selection pressure for the genetic component in any of the N loci (e.g. placing one or more positive selection markers close to said genetic component). Then the events bearing all the N loci will be strongly privileged in the segregation.

### 1.5. Containment of Genetic Flux

The invention can also be applied to limit the potential propagation of genetic components through sexual crossing by using the linkages hereabove:
(i) linking one or more Toxic Nuclease(s) to the genetic locus, or the genetic component to be contained, e.g. by placing the gene(s) coding for said Toxic Nuclease(s) into said locus or next to said genetic component—and/or
(ii) linking one or more corresponding Inhibitor(s) to a locus (e.g. by placing the gene(s) coding for said Inhibitor(s) into said locus or next to said genetic component) that normally segregates from the locus to be contained during sexual crossing, an example of which is the homolog (i.e. same chromosomal location on the other chromosome of the same pair) of the locus to be contained in a diploid cell or organism.

After sexual crossing, the events bearing the genetic locus to be contained will bear the Toxic Nuclease(s) without the corresponding Inhibitors, and will thus not propagate.

### 1.6. Containment of Genetic Flux Through Hybridization

The invention can also be applied to limit the potential propagation of genetic components through hybridization by linking chromosomes from one donor event together:
(i) using the approach hereabove, a genetic component which flux is to be contained can be linked to (one or more of) its homolog chromosome(s) so that, should these two (or more) chromosomes be together in an event achieved through hybridization with an external variant/cell/organism, then the next generation of said event, reverting to normal ploïdy will only be able to bear said genetic component together with its original homolog chromosome(s);
(ii) likewise, such genetic component which flux is to be contained can also be linked to loci on all the chromosomes of the event it is in so that segregation through hybridization would be subject to Toxic Effect.

### 1.7. “Suicide” Chromosome

The invention can also be applied to limit the potential propagation of a chromosome/plasmid outside of a chosen genetic context by:
(i) using one or more Toxic Nuclease(s) targeting loci that are all located on a given chromosome/plasmid, and which corresponding inhibitors are on selected loci to which said chromosome is to be linked. Said Toxic Nuclease(s) can be linked to multiple loci on said chromosome/plasmid (e.g. copies of genes coding for said Toxic Nuclease(s) can be placed in multiple locations along the chromosome/plasmid) and their specific targets can also be chosen (or engineered) to be in multiple loci of said chromosome/plasmid.

### 2. ORGANELLE SELECTION

### 2.1. Homoplasmy

Toxic Nuclease(s) can also be designed to bias the composition of the organelles present in a cell or organism. An instance is to achieve homoplasmy, which is usually obtained through strong positive selection for an engineered organelle bearing a marker. To achieve homoplasmy in a cell bearing more than one genetic representative of a given organelle, one or more Toxic Nucleases, specifically targeting the unwanted alleles of said organelles can be used to counter select them. One instance where such an approach can be implemented is when said organelle was engineered or selected to evade the effect of said Toxic Nucleases (e.g. genome clear of targetable site(s) by the Toxic Nuclease that will destroy a critical component in the other organelles—such as polymerase catalytic site).

### 2.2. Selection for Organelle Combinations

The linkage invention can also be applied to organelles. By choosing the cross-linked (or circularly-linked) loci to be in different organelles, any cell or organisms not having all such different organelles will not propagate. An example being the cross linkage described hereabove where the First Locus is in the genome of one organelle and the second Locus is in the genome of another organelle. The loss of any of the two organelles (e.g. through homoplasmy) will result in a cell or organism that does not propagate.

### 2.3. Endo-Symbiont Stabilization

The same invention applies to endo-symbionts, the genome of which usually replicates independently from that of its host. By choosing the cross-linked (or circularly-linked) loci to be in different partners of the endo-symbiosys, any cell or organisms not having all such different partners will not propagate. An example being the cross linkage described hereabove where the First Locus is in the genome of one endo-symbiont and the second Locus is in the genome of another endo-symbiont. The loss of any of the two endo-symbiont will result in a cell or organism that does not propagate.

### 3. SELECTIVE CELLS/TISSUE DEPLETION

The invention may also be used in combination with non-constitutive expression of either or both the Toxic Nuclease(s) or the corresponding Inhibitor(s).

### 3.1. Selection for Co-Expression

There are possible uses of the invention to prevent the formation of selected cell/tissue types. Toxic Nucleases or corresponding inhibitors can be made active only in selected cellular/tissular contexts. Only the tissue/cell types where no Toxic Nuclease is expressed or all expressed Toxic Nuclease(s) are in presence of expressed corresponding Inhibitors will survive. Linking the expression of pairs of Toxic Nucleases and corresponding Inhibitors (as in 1.3 or 1.4) to the expression of selected genetic components each expressed in specific cellular/tissular contexts, will result in the selection of tissue/cell types that co-express said selected genetic components.

### 3.2. Sterility

In addition to the approach described hereabove (section 1.5) there are possible uses of the invention to prevent the formation of functional gametes. Toxic Nucleases or corresponding inhibitors can be made active only in selected cellular/tissular contexts. By making a Toxic Nuclease expressed only upon gamete differentiation, one can prevent such differentiation. Another approach lies in the constitutive presence of a Toxic Nuclease and the constitutive presence of an Inhibitor, except in gametes. Alternatively, both approaches can be combined.

### 3.3. Morphology

Likewise, the selective depletion of a tissue/cell type (other than gametes) or cells in a particular phase, can be achieved through the same approaches, affecting morphology for example.

### 3.4. Biasing Chromosome Silencing

During development, some species undergo selective chromosome silencing, an example of which is X chromosome silencing in mammalian females. One can bias which chromosome will be silenced in an individual/event or in parts thereof (selected tissue/cell types) by linking one or more Toxic Nuclease(s) to genetic components on said chromosome to be silenced in a way such that said Toxic Nuclease(s) are expressed/present when said chromosome is not silenced, and linking corresponding Inhibitor(s) to loci on other chromosomes where their expression/presence will not take place in the conditions (e.g. cell/tissue type) where chromosome silencing bias is desired.

### 4. SELECTION FOR EPIGENETIC STATUS

### 4.1. Selection of Methylated or Unmethylated Status

The invention can also be applied using Toxic Nucleases that are differentially sensitive to chromatin status, thereby acting only upon specific epigenetic conditions. An example of implementation lies in differential sensitivity to DNA methylation. Toxic Nuclease(s) targeting critical site(s) in the genome that can be subject to methylation will act only on unmethylated DNA, or, reciprocally only on methylated DNA. Differentially expressed Inhibitor(s) can be used to prevent the action of the Toxic Nucleases in irrelevant tissues or cell phases. Said Inhibitors not being present in the cell or tissue when said Toxic Nuclease is to act differentially on methylated or unmethylated DNA.

1. Method for segregating genes in a progeny cell comprising the steps of:
a) Introducing into the genome of a progenitor cell, at a first locus, a gene encoding a nuclease component having a genotoxic effect on said cell;
b) Introducing at the same locus or at a second locus, a gene encoding a nuclease inhibitor, the expression of which inhibits the genotoxic effect of said nuclease component;
c) Cultivate the transformed cells of step a) as to obtain progeny cells that include said first and second loci into their genomes.

2. Method according to claim 1, wherein said progenitor cells are gametes having undertaken meiosis and fecundation with other gametes between steps b) and c).

3. Method according to claim 2, wherein several genes are present at said first and second loci, so that those genes can segregate together.

4. Method according to any one of claims 1 to 3, wherein said method includes introducing further genes at various loci encoding nuclease components.

5. Method according to claim 4, wherein said further genes encode N different nuclease components, whereas N genes encoding nuclease inhibitors respectively inhibiting those nuclease components are also introduced into the cell.

6. Method according to any one of claims 1 to 5, wherein said progeny cell is a plant cell or a protoplast that is grown into an adult plant.

7. Method according to any one of claims 1 to 5, wherein the genome of said progeny cell is injected into an embryo for producing a transgenic animal.

8. Method for producing non-human gametes containing a desired combination of alleles comprising the steps of:
a) producing a transgenic animal according to the method of claim 7;
b) growing said transgenic animal in order it to produce gametes;
c) collecting the living gametes produced by said transgenic animal, which mainly contain the segregated loci.

9. Method according to claim 8, wherein the living gametes contain mainly the chromosome X, or mainly the chromosome Y.

10. Method for producing a male or female non-human animal by fertilizing the gametes obtained by the method of claim 9.

11. Method for obtaining a sterile organism or genetic containment of a locus in the genome of a living organism comprising the steps of
a) linking said locus to a gene encoding a nuclease component having a genotoxic effect on said living organism;
b) linking the allele of said locus to a gene encoding an inhibitor specific to said nuclease component;
c) so that when crossing this living organism genome with the genome of another living organism the nuclease component thereof induces toxicity in the progeny cells.

12. Method for selection of cells having a defined organelle(s) composition comprising the steps of:
a) Introducing a gene(s) encoding a nuclease component(s) into the genetic material of a desired organelle(s), said nuclease component(s) having a genotoxic effect on the genome of the cell that host said desired organelle(s);
b) Introducing into the cell's genome a gene(s) encoding an inhibitor(s) specific to said nuclease component(s);
c) Culturing said cell to obtain cells containing the selected organelle(s).

13. Method for the selection of cells comprising endosymbiont(s)
a) Introducing a gene(s) encoding a nuclease component(s) into the genetic material of a desired endosymbiont(s), said nuclease component(s) having a genotoxic effect on the genome of the cell that host said desired endosymbiont(s);
b) Introducing into the cell's genome a gene(s) encoding an inhibitor(s) specific to said nuclease component(s);
c) Culturing said cell to obtain cells containing the selected endosymbiont(s).

14. Method according to claim 13, wherein the genome of the cell or of the endosymbiont(s) is crossed with the genome of another cell(s).

15. Method according to any one of claims 1 to 14, wherein said nuclease toxic component is a specific endonuclease.

16. Method according to claim 15, wherein said specific endonuclease(s) is(are) directed against sequence(s) that is repeated into the genome.

17. Method according to claim 15 or 16, wherein said specific endonuclease(s) is(are) directed against sequence(s) that are non-coding genomic regions.

18. Method according to claim 15, wherein said endonuclease targets genes involved in the motility or the functionality of the gametes.

19. Method according to any one of claims 15 to 17, wherein said endonuclease is a rare-cutting endonuclease selected from a homing endonuclease, a TAL-nuclease, a MBBBED-nuclease, or a zing-finger nuclease (ZFN).

20. Method according to any one of claims 1 to 17, wherein said nuclease inhibitor is an antagonistic ligand, an antibody, an interfering polynucleotide.

21. Method according to any one of claims 1 to 17, wherein said nuclease component is NucA and said inhibitor is NuiA.

22. Method according to any one of claims 1 to 17, wherein said nuclease component is CoIE7 and said inhibitor is Im7.

23. Method according to any one of claims 1 to 17, wherein said nuclease component comprises Cas9 and said inhibitor is an anti-guide-RNA.

24. A non-human cell or gamete comprising in its genome an exogenous gene encoding a nuclease component and another exogenous gene encoding a nuclease inhibitor directed against said nuclease component.

25. A non-human cell or gamete according to claim 24, wherein said exogenous genes encoding a nuclease component and inhibitor thereof are inserted on different loci.

26. A non-human cell or gamete according to claim 24, wherein said exogenous genes encoding a nuclease component and inhibitor thereof are inserted on alleles encoding homologous genes.

27. A genetic construct comprising one or more genes of interest located between a first gene encoding a nuclease toxic component and a second gene encoding a specific nuclease inhibitor.

28. A genetic construct according to claim 27, further comprising a gene encoding a selectable marker or a selection gene.

29. A genetic construct according to claim 27 or 28, further comprising an exogenous promoter to activate the expression of said first and/or second genes encoding a nuclease component and specific nuclease inhibitor.

30. A genetic construct according to any one of claims 27 to 29, further comprising cleavage sites for a rare-cutting endonuclease.

31. A genetic construct according to claim 30, wherein said cleavage sites are located in the first gene encoding the nuclease component or on both sides of said gene to knock out or remove said first gene from the genome of the progeny cell.

32. A set of two genetic constructs, wherein the first genetic construct comprises a gene encoding a nuclease component and the second construct comprises a gene encoding a nuclease inhibitor specific to the nuclease component encoded by the first genetic construct.

33. A set of two genetic constructs according to claim 32, wherein the first and second genetic constructs further comprise genes of interest to create a genetic linkage between them.

34. A set of N genetic constructs each comprising one or several genes of interest located between a first gene encoding a nuclease component and a second gene encoding a specific nuclease inhibitor, wherein said genes encoding a nuclease inhibitor are different.

35. A set of N genetic constructs, wherein said nuclease inhibitor of the N-1 genetic construct is directed against the nuclease toxic component of the N genetic construct.

36. A set of N genetic constructs according to claim 35, wherein N=2.

37. A set of N genetic constructs according to claim 35, wherein N>2.

38. Genetic construct(s) according to any of claims 27 to 37, wherein said nuclease toxic component is a specific endonuclease.

39. Genetic construct(s) according to claim 38, wherein said specific endonuclease(s) is (are) directed against sequence(s) that is repeated into the genome.

40. Genetic construct(s) according to claim 38 or 39, wherein said specific endonuclease(s) is(are) directed against sequence(s) that are non-coding genomic regions.

41. Genetic construct(s) according to any of claims 35 to 40, wherein said endonuclease target genes involved in the functionality of the gametes.

42. Genetic construct(s) according to any of claims 35 to 41, wherein said endonuclease is a rare-cutting endonuclease selected from a homing endonuclease, a TAL-nuclease, a MBBBD-nuclease, or a zing-finger nuclease (ZFN).

43. Genetic construct(s) according to any of claims 35 to 42, wherein said nuclease inhibitor is an antagonistic ligand, an antibody, an interfering polynucleotide.

44. Genetic construct(s) according to any of claims 35 to 43, wherein said nuclease component is NucA and said inhibitor is NuiA.

45. Genetic construct(s) according to any of claims 35 to 44, wherein said nuclease component is CoIE7 and said inhibitor is Im7.

46. Genetic construct(s) according to any of claims 35 to 45, wherein said nuclease component comprises Cas9 and said inhibitor is an anti-guide-RNA.

47. Genetic construct(s) according to any of claims 35 to 45, wherein the genetic constructs are inserted in a cell or gamete genome.

48. Genetic construct(s) according to any of claims 35 to 45, wherein the genetic constructs are formed in a cell genome.
